(19) United States
(12) Patent Application Publication
(10) Pub. No.: US 2024/0366876 A1
(43) Pub. Date: Nov. 7, 2024

(54) APPLICATOR TIPS USEFUL FOR SURGICAL APPLICATION OF A VISCOUS SUBSTANCE

(71) Applicant: Cook Biotech Incorporated, West Lafayette, IN (US)

(72) Inventors: Cody L. Ruck, Lafayette, IN (US); William F. Hoggatt, West Lafayette, IN (US); Loran D. Solorio, West Lafayette, IN (US)

(73) Assignee: Cook Biotech Incorporated, West Lafayette, IN (US)

(21) Appl. No.: 18/646,026

(22) Filed: Apr. 25, 2024

Related U.S. Application Data
(60) Provisional application No. 63/463,677, filed on May 3, 2023.

(51) Int. Cl. A61M 5/31; A61M 35/00

(52) U.S. Cl. CPC A61M 5/3134; A61M 35/003

(57) ABSTRACT

The present disclosure provides medical products and methods including an applicator tip for connection to a syringe. The applicator tip is advantageous when dispensing viscous fluids, such as adhesives and/or sealants.

### BACKGROUND

The present disclosure relates generally to the field of instruments for dispensing flowable material for use in a variety of medical procedures.

As further background, medical procedures often require application of viscous fluids, such as adhesives and sealants, to patient tissue or other surfaces. Such fluids are sometimes dispensed from a syringe or similar device. Prior known techniques and devices often suffer from complexity in manufacture or use and/or result in an undesirable distribution of the material on the target surface.

There remain needs for improved or alternative devices and methods for the delivery of fluidic medical substances onto patient tissue or other surfaces. Aspects of the present disclosure are addressed to these needs.

### SUMMARY

In some aspects, provided are devices and systems useful for dispensing a fluid. In accordance with some forms of the disclosure, such devices are configured to facilitate the application of a viscous fluid unto a substrate. Accordingly, in one embodiment the present disclosure provides an applicator tip for connection to a syringe barrel having a distal end including a cannulated spout spaced from and surrounded by a connector having an internal connector thread on an internal wall of the connector. The applicator tip comprises a tip body defining a proximal opening, a dispensing opening spaced from the proximal opening, and a tip body lumen extending from the proximal opening to the dispensing opening. The tip body further includes a proximal coupling portion defining an exterior thread cooperable with the internal connector thread of the syringe barrel to establish a threadedly attached condition of the applicator tip to the syringe barrel in which the tip body lumen is in fluid communication with an output opening of the cannulated spout. The tip body lumen is defined by an interior lumen wall, and the interior lumen wall includes a sealing surface portion configured to contact and seal against an exterior surface of the cannulated spout in the threadedly attached condition. In accordance with certain inventive variants, the dispensing opening is a slot, optionally a trapezoidal slot. In one embodiment, the tip body lumen includes a distal lumen portion having a longitudinal segment having a decreasing cross-sectional area as it extends distally. In some forms, the tip body includes a distal application portion having a first exterior sidewall portion defining at least one surface that is convexly curved in a longitudinal direction along the distal application portion, optionally the distal application portion further has a second exterior sidewall portion opposite the first exterior sidewall portion and defines at least one surface that is concavely curved in the longitudinal direction along the distal application portion. In certain modes, the interior lumen wall defines a decreasing lumen diameter in a distal direction that provides said sealing surface portion. In one aspect the longitudinal segment has an increasing cross-sectional area extends to the dispensing opening. In certain embodiments, the tip body lumen includes a proximal coupling lumen portion within the proximal coupling portion that is substantially circular in cross-section and has a longitudinal axis. In some forms, the longitudinal axis of the proximal coupling lumen portion intersects the dispensing opening. In certain embodiments, the tip body is composed of a material impenetrable to visible light and/or ultraviolet light. In some aspects, a distal region of the tip body defines a fluid control member comprising a first sidewall opposite a second sidewall, and an upper wall extending between the first sidewall and the second sidewall, the upper wall opposite an open side of the fluid control member. In some forms, the fluid control member is configured for positioning a longitudinally extending edge of the first sidewall and a longitudinally extending edge of the second sidewall against a target surface such that the target surface, first sidewall, second sidewall and upper wall together define a composite longitudinal lumen for receiving a bead of a fluid dispensed through the tip body. In some forms, the present disclosure provides a product comprising a syringe barrel and an applicator tip as described above threadedly connected to or threadedly connectable to the syringe barrel to establish the threadedly connected condition.

In another embodiment, the present disclosure provides a product comprising a syringe barrel having a distal end including a cannulated spout, an applicator tip threadedly connected to or threadedly connectable to the syringe body. The tip body defining a proximal opening, a dispensing opening spaced from the proximal opening, and a tip body lumen extending from the proximal opening to the dispensing opening. The tip body having an interior lumen wall defining the tip body lumen. Wherein the syringe barrel and tip body are connected or connectable such that the tip body lumen is in fluid communication with a distal end opening of the cannulated spout and in which a sealing surface portion of the interior lumen wall is in contact with and sealed against an exterior side surface of the cannulated spout. In some embodiments, the cannulated spout includes a distally-tapering cone. In some forms, the distal end of the syringe barrel includes a connector having connector threads on an internal wall thereof, with the cannulated spout being spaced from and surrounded by the connector, and the tip body includes a proximal coupling portion defining an exterior thread that is threadably cooperable with the connector threads. In certain modes, the tip body lumen includes a distal lumen portion having a decreasing cross-sectional area as it extends distally. In one aspect, the sealing surface occurs on a portion of the interior lumen walls that define a decreasing lumen diameter in a distal direction, optionally wherein the sealing surface occurs distal to the proximal coupling portion of the tip body. Certain variants include a distal application portion having an exterior sidewall portion defining at least one surface that is convexly curved in a longitudinal direction along the distal application portion. In certain embodiments, the dispensing opening is a slot, optionally a trapezoidal slot. In one aspect, the longitudinal axis of the proximal coupling portion intersects the dispensing opening. In some forms, the tip body is composed of a material impenetrable to visible light and/or ultraviolet light. In certain aspects, the tip body lumen is the only lumen through the tip body. In accordance with some forms, the tip body defines a fluid control member comprising a first sidewall opposite a second sidewall, and an upper wall extending between the first sidewall and the second sidewall, the upper wall opposite an open side of the fluid control member.

In another embodiment, the present disclosure provides an applicator tip for dispensing a viscous material, the applicator tip comprising a tip body defining a proximal opening, a dispensing opening spaced from the proximal opening, and a tip body lumen extending from the proximal opening to the dispensing opening. The tip body has a distal region that defines a fluid control member comprising a first sidewall opposite a second sidewall, and an upper wall extending between the first sidewall and the second sidewall, the upper wall opposite an open side of the fluid control member. In certain embodiments, the fluid control member is configured for positioning a longitudinally extending edge of the first sidewall and a longitudinally extending edge of the second sidewall against a target surface such that the target surface, first sidewall, second sidewall and upper wall together define a composite longitudinal lumen for receiving a bead of a fluid dispensed through the tip body. In certain embodiments, the tip body lumen is defined by a tip body lumen wall, and wherein the upper wall has an inner surface contiguous with the tip body lumen wall and extending to the dispensing opening, and wherein the inner surface is a smooth surface. In some forms, the tip body lumen comprises a coupling lumen portion having a substantially circular cross-sectional area. In one embodiment, the interior lumen wall defines a decreasing lumen diameter in a distal direction that provides a sealing surface portion. In some modes, the tip body is composed of a material impenetrable to visible light and/or ultraviolet light. In certain aspects, the tip body includes a distal application portion having a first exterior sidewall portion defining at least one surface that is convexly curved in a longitudinal direction along the distal application portion.

In another embodiment, the disclosure provides a product, comprising: a syringe barrel having a distal end with a cannulated spout, and an applicator tip as described above connected to or connectable to the syringe barrel to establish a connected condition in which the tip body lumen is in fluid communication with an opening of the cannulated spout. In some forms, the product is an assembled apparatus in which the applicator tip is in the connected condition. In other forms, the product is a kit including the syringe barrel and the applicator tip not in the connected condition. In certain embodiments the product is contained within a sterile sealed package.

In another embodiment, the present disclosure provides a method for assembling an apparatus for dispensing a flowable material. The method comprises connecting an applicator tip as described above to a syringe barrel or to an extension tube connected to a syringe barrel.

In another embodiment, the present disclosure proves a method for dispensing a flowable material. The method comprises providing a product as described above, or an apparatus assembled as described above, and causing the flowable material to flow from the syringe barrel and out of the dispensing opening. In certain embodiments, such methods comprise advancing a plunger within a chamber defined by the syringe barrel.

In another embodiment, the present disclosure provides a medical kit for administration of a flowable material to a patient. The medical kit comprising: a flowable material for administration to a patient, or one or more precursor materials for preparing a flowable material for administration to a patient, and an applicator tip as described above, or a product as described above. In accordance with some forms, the flowable material is a photocurable sealant or photocurable adhesive material.

Still further embodiments, as well as features and advantages of embodiments described herein, will be apparent to persons skilled in the relevant field from the descriptions herein.

### BRIEF DESCRIPTION OF THE DRAWINGS

FIG. 1 provides a perspective view of one embodiment of an applicator tip of the present disclosure.

FIG. 2 provides a back view of one embodiment of an applicator tip of the present disclosure.

FIG. 3 provides a front view of one embodiment of an applicator tip of the present disclosure.

FIG. 4 provides a cross-sectional view of one embodiment of an applicator tip of the present disclosure.

FIG. 5 provides a cross-sectional view of one embodiment of an apparatus of the present disclosure.

FIG. 6 provides a perspective view of one embodiment of an applicator tip of the present disclosure.

FIG. 7 provides a back view of one embodiment of an applicator tip of the present disclosure.

FIG. 8 provides a front view of one embodiment of an applicator tip of the present disclosure.

FIG. 9 provides a cross-sectional view of one embodiment of an applicator tip of the present disclosure.

FIG. 10 provides a cross-sectional view of one embodiment of an apparatus of the present disclosure.

FIG. 11 provides a cross-section view of one embodiment of an apparatus of the present disclosure.

FIG. 12 provides an expanded cross-sectional view of a portion of one embodiment of an applicator tip of the present disclosure.

FIG. 13 provides a cross-sectional view of an apparatus of the present disclosure.

### DETAILED DESCRIPTION

Reference will now be made to certain embodiments, some of which are illustrated in the drawings, and specific language will be used to describe the same. It will nevertheless be understood that no limitation of the scope of the disclosure is thereby intended. Any alterations and further modifications in the described embodiments and any further applications of the principles as described herein are contemplated as would normally occur to one skilled in the art to which this disclosure relates.

As used herein, the term “proximal” means closer to the operator and the term “distal” means further away from the operator.

As disclosed above, aspects of the present disclosure relate to applicator tips. In certain embodiments, the present disclosure relates to applicator tips for use with a syringe. Applicators tips of the present disclosure may be adapted to work with any suitable syringe. In general, a syringe comprises a barrel having an internal volume and a plunger movable through the barrel to force a fluid from the internal volume through a distal spout. The distal end of a syringe may be adapted for connection to a dispensing device, such as an applicator tip as disclosed herein. In some forms, the distal end of a syringe includes a cannulated seal element. Such cannulated seal elements generally comprise an elongate protrusion extending from the distal end of the syringe barrel, the distal spout is positioned at or near the distal end of the cannulated seal element. In some forms, the cannulated seal element is configured to create a fluid seal with a dispensing device, such as an applicator tip, such that fluid dispensed form the distal tip is transferred to the dispensing device. In accordance with some forms, the cannulated seal element may have a tubular or conical form. In certain embodiments, the cannulated seal element is a distally-tapering cone. In addition to the cannulated seal element, the distal end of a syringe may be adapted to secure a dispensing device, for example with a threaded attachment. In this way, a cylinder having internal threads may be present surrounding, or partially surrounding, the cannulated seal element. In certain embodiments, the syringe distal tip comprises a luer tip.

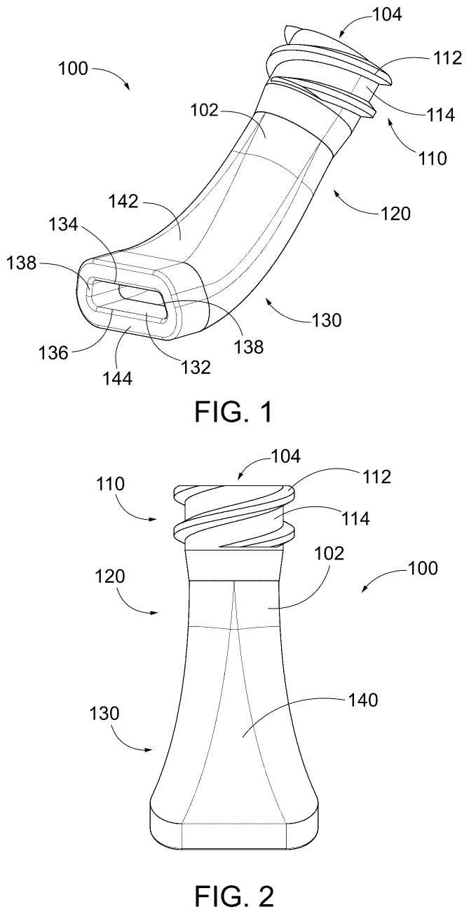

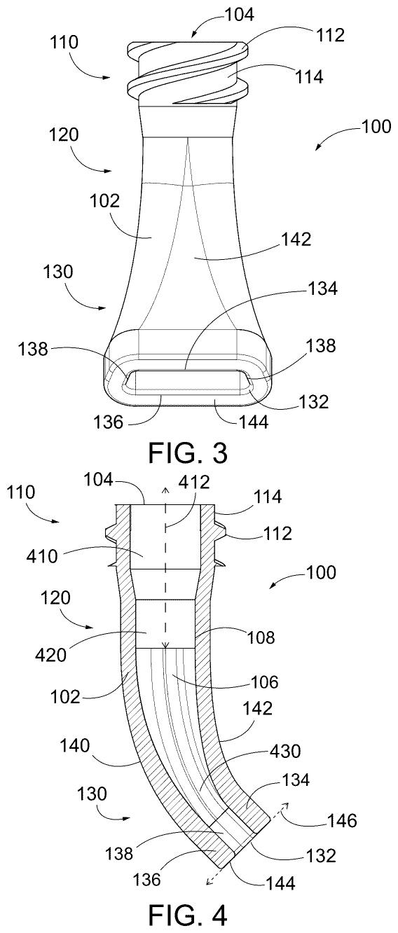

With reference to the embodiment illustrated in FIGS. 1-4, shown is applicator tip 100. Applicator tip 100 includes a tip body 102 having a proximal input opening 104, a dispensing opening 132 spaced from the proximal input opening 104, and a tip body lumen 106 extending between the proximal input opening 104 and the dispensing opening 132. Tip body lumen 106 is defined by an overall tip body lumen wall 108 bounding the lumen 106. In the illustrated embodiment, applicator tip 100 includes a proximal coupling portion 110, a tubular shaft portion 120 extending distally from the proximal coupling portion 110, and a distal application portion 130 extending distally from the tubular shaft portion 120. Threads 112 are positioned on external surface 114 of proximal coupling portion 110 and are configured for threaded attachment to corresponding threads of a syringe barrel. In the illustrated embodiment threads 112 are positioned on an external, or convex, surface of syringe tip 100. Such a configuration is advantageous for threaded attachment to syringe barrel threads positioned on an internal, or concave, surface such as those described above. It is within the scope of aspects of the present disclosure to provide applicator tips having threads positioned on an internal, or concave, surface preferably an internal surface of the proximal coupling surface. Such a configuration is advantageous for threaded attachment to syringe barrel threads positioned on external, or convex, surfaces.

FIG. 4 is a cross-sectional view of the applicator tip 100 illustrated in FIGS. 1-3. In the illustrated embodiment the tip body lumen 106 provides a fluid path that extends from proximal opening 104, through coupling lumen portion 410, shaft lumen portion 420, distal application lumen portion 430, and to dispensing opening 132. Dispensing opening 132 is defined by upper wall 134, sidewalls 138, and lower wall 136. In the illustrated embodiment, coupling lumen portion 410 has a longitudinal axis 412. In accordance with some forms, the tip body 102 is configured to align the longitudinal axis 412 of coupling lumen portion 410 with a longitudinal axis of the distal cannulated spout of the syringe. Shaft lumen portion 420 is distal of and in fluid communication with proximal coupling lumen portion 410. Distal application lumen portion 430 is distal of and in fluid communication with shaft lumen portion 420. In certain embodiments the tip body 102 defines only a single continuous and unbranched passageway from the proximal input opening 104 to the dispensing opening 132, provided by the tip body lumen 106.

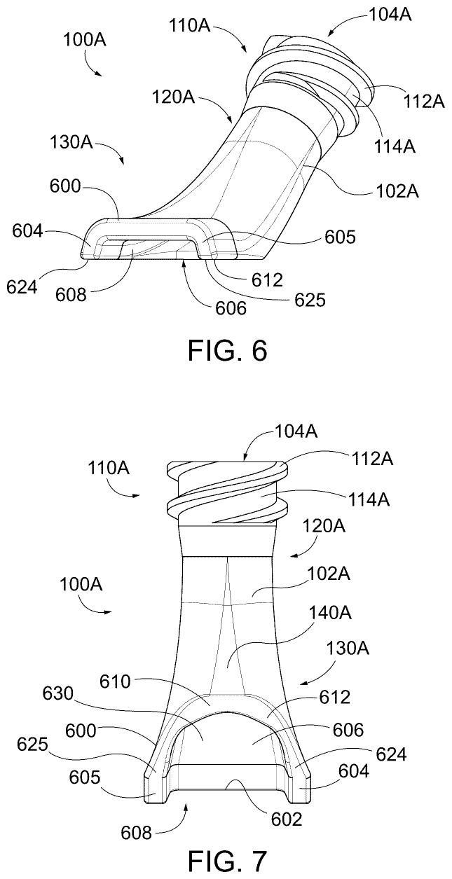

Tip bodies 102 of the present disclosure may be curved. For example, in accordance with some forms distal application portion 130 includes a first exterior sidewall portion 140 defining at least one surface that is convexly curved with respect to longitudinal axis 412. In certain embodiments, distal application portion 130 includes a second exterior sidewall portion 142 defining at least one surface that is concavely curved with respect to longitudinal axis 412. In the illustrated embodiment, first exterior sidewall portion 140 is opposite second exterior sidewall portion. Thus, in accordance with some forms, longitudinal axis 412 intersects with an interior sidewall of the distal application portion 130. In alternative embodiments, such as that illustrated in FIGS. 6-11, longitudinal axis 412 intersects with dispensing opening 132. In the illustrated embodiment, the curvature of the distal application portion causes distal end edge surface 144 to extend in a plane 146 oblique to the longitudinal axis 412 of coupling lumen portion 410.

Tip body lumens 106 of the present disclosure may also comprise lumen portions of varying shape and cross-sectional area. For example, in certain embodiments the cross-sectional shape of coupling lumen portion 410 and/or the shaft lumen portion 420 is generally circular. In certain embodiments dispensing opening 132 has an elongate cross-sectional shape, such as a slot. In certain embodiments, dispensing opening 132 has a trapezoidal cross-sectional shape. Thus, the tip body lumen 106, for example including coupling lumen portion 410, shaft lumen portion 420, and distal application lumen portion 430, can in some forms transition from a circular cross-sectional shape to a trapezoidal cross section shape.

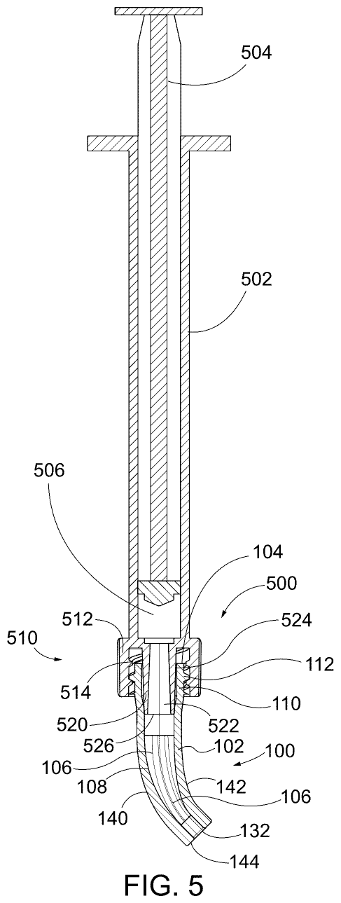

With reference to FIG. 5, shown is one embodiment of an apparatus 500 as provided by the present disclosure. Apparatus 500 comprises syringe barrel 502 having distal end 510, and a syringe plunger 504. In the illustrated embodiment, the distal end 510 of the syringe barrel comprises a connector 512, such as a cylinder, having internal threads 514 on an internal wall of connector 512, and a cannulated spout 520 spaced from and surrounded by such internal wall. Cannulated spout 520 defines cannula 522 and an exterior side surface 524. Cannulated spout 520 extends to distal end opening 526 of spout 520. Cannula 522 is in fluid communication with syringe barrel lumen 506. Products disclosed herein comprise any applicator tip disclosed herein operably connected to, or operably connectable to, a syringe or other fluid dispensing apparatus. In accordance with some forms, the proximal coupling portion 110 of tip body 102 is received between cannulated spout 520 and the internal wall of connector 512 that defines the internal threads 514. In certain embodiments, in the connected condition of the applicator tip 100 and the syringe barrel 502, the exterior side surface 524 of the cannulated spout 520 contacts and seals against the interior wall 108 of tip body 102 that defines tip body lumen 106. For example, in other forms of an apparatus, an extension tube may be positioned between the applicator tip 100 and the syringe barrel 502 and threadedly connected to each. Use of an extension tube may allow for greater flexibility in maneuvering the applicator tip for application of the flowable material to a patient tissue, for example through another elongate cannulated surgical instrument such as a trocar.

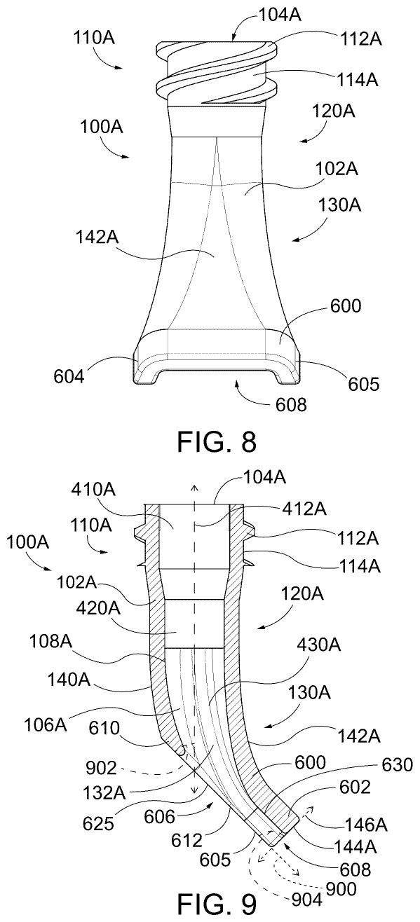

Turning now to the embodiment illustrated in FIGS. 8-11, the illustrated embodiment includes many of the features detailed above for tip 100, which are labeled with corresponding numbers followed by “A”. Briefly, applicator tip 100A comprises a tip body lumen 106A extending from proximal opening 104A to dispensing opening 132A. Dispensing opening 132A comprises bottom opening 606 and anterior opening 608. Applicator tip 100A comprises proximal coupling portion 110A defining coupling lumen portion 410A internally, and having threads 112A positioned on an external surface 114A. Tubular shaft portion 120A defines shaft lumen portion 420A. Distal application portion 130A defines dispensing application lumen portion 430A. In the illustrated embodiments, central lumen coupling axis 412A extends longitudinally through dispensing opening 132A.

The illustrated embodiments include fluid control member 600 positioned at the distal end of applicator tip 100A. Fluid control member 600 comprises an elongate upper wall 602, elongate side walls 604 and 605, bottom opening 606, and anterior opening 608. Bottom opening 606 is defined by elongate side walls 604 and 605, posterior wall 610, and anterior opening 608. As shown in FIG. 7, rear wall 610 comprises an arcuate wall extending from sidewall 604 to sidewall 605. In some forms, flat contact surface 612 is formed by posterior wall 610, longitudinally extending edge 624 of side wall 604, and longitudinally extending edge 625 of side wall 605. In certain embodiments, flat contact surface 612 extends in a plane 900. In accordance with some forms angle 902 is formed between plane 900 and longitudinal axis 412A. As disclosed herein, longitudinal axis 412A may be aligned with a longitudinal axis of syringe barrel 502A. In some forms, angle 902 is 15° to 75°, preferably 20° to 60°, more preferably 25° to 50°. In some embodiments, angle 904 is formed between plane 900 and 146A. In some forms, angle 904 is 15° to 75°, preferably 25° to 65°, more preferably 35° to 55°.

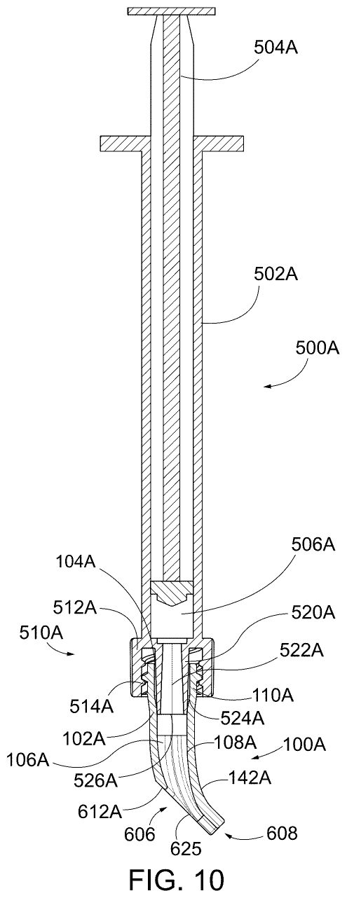

With reference to FIG. 10, shown is one embodiment of an apparatus 500A as provided by the present disclosure. Apparatus 500A comprises syringe barrel 502A having distal end 510A, and syringe plunger 504A. In the illustrated embodiment, distal end 510A comprises connector 512A having internal threads 514A, and cannulated spout 520A defining cannula 522A. Cannulated spout 520A defines cannula 522A and an exterior side surface 524A. Cannulated spout 520A extends to distal end opening 526A of spout 520A. Cannula 522A is in fluid communication with syringe barrel lumen 506A. Such systems as disclosed herein comprise any applicator tip disclosed herein operably connected to a syringe, or other fluid dispensing apparatus. In accordance with some forms, the proximal coupling portion 110A of applicator tip 100A is received between cannulated spout 520A and connector 512A. In certain embodiments, an exterior surface 524A of the cannulated spout 520A contacts and seals against the interior walls 108A of applicator tip 100A. In alternative forms, an extension tube may be threaded to an applicator tip. Use of an extension tube may allow for greater flexibility in maneuvering the applicator tip. An extension tube for use with the present disclosure comprises any suitable length of tubing capable of facilitating passage of a fluid into the applicator tip.

Thus, in certain embodiments applicator tips as disclosed herein may include a fluid control member. In general, a fluid control member comprises an extension of the body wall of the upper and side wall portions of the distal application portion. In certain embodiments the dispensing opening is enlarged to include a bottom opening. In some forms the applicator tip is configured for contact with a substrate, for example certain embodiments include a flat contact surface surrounding part of the bottom opening. Such a configuration allows for dispensing a fluid through the applicator tip and onto a substrate while the fluid is still within the lumen formed by the fluid control member and the substrate. The dispensed fluid is then bound by the substrate, the posterior wall, the elongate sidewalls and the upper wall of the fluid control member. As the applicator tip is drawn in a direction away from the anterior opening the fluid is released through the anterior opening. In this way, a more uniform and controlled fluid dispersal pattern can be achieved. Particular advantage is found when dispensing viscous fluids. Top wall of the fluid control member operates to smooth and compress the fluid already on the substrate but within the composite longitudinal lumen. Such compression is operable to reduce or eliminate air gaps between the viscous fluid and the substrate and to create a smooth layer of the dispensed fluid. In accordance with some forms, top wall of the fluid control member has a smooth inner surface 630 which is contiguous with the tip body lumen wall surface 108A and extends to the dispensing opening. In accordance with such embodiments, the upper wall has no overhangs, ledges, or grooves which would impede fluid flow.

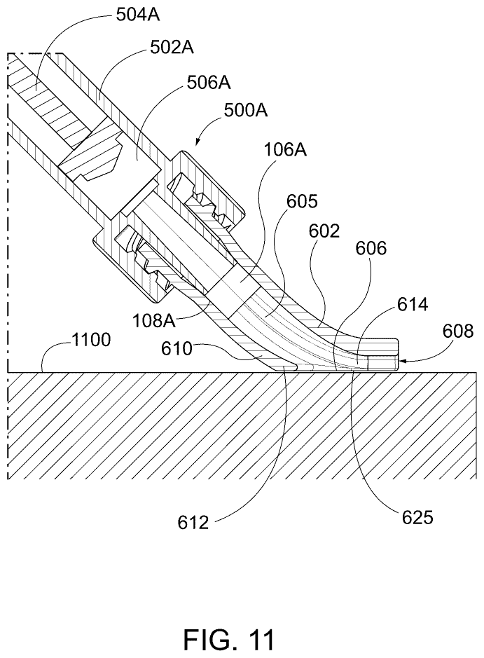

FIG. 11 illustrates one embodiment of an apparatus 500A as described herein positioned against a substrate 1100. In the illustrated embodiment, contact surface 612 is pressed against substrate 1100. Composite longitudinal lumen 614 is formed between substrate 1100, upper wall 602, side wall 605, side wall 604, and posterior wall 610. Composite longitudinal lumen 614 forms a fluid passage between distal application lumen portion 430 and anterior opening 608.

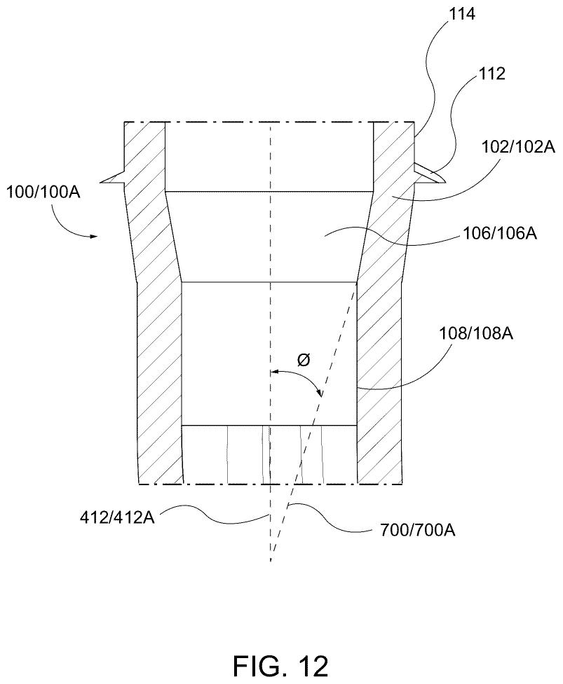

As detailed herein, tip body lumen 106/106A may comprise lumen portions of varying shape and cross-sectional area. Transition(s) of the shape and/or cross-sectional size of the tip body lumen 106/106A can be configured to avoid any proximally-facing wall surfaces angled at ninety degrees to the direction of fluid flow through the lumen 106/106A. This can facilitate avoiding resistance to fluid flow from the proximal input opening 104/104A to the dispensing opening 132/132A through the tip body lumen 106/106A. In some forms, longitudinal transitions of the cross-sectional size and/or shape of the tip body lumen 106/106A are provided by lumen wall 108/108A. In such transitions, the total cross-sectional area of any portion of the tip body lumen may increase or decrease along a longitudinal segment of the tip body lumen 106/106A as it extends distally. In certain embodiments, lumen wall 108/108A include a sealing surface portion configured to contact and seal against an exterior surface of a cannulated spout inserted through proximal opening 104/104A. In some forms, sealing surface portion comprises a longitudinal portion of lumen wall 108/108A wherein the wall tapers towards the longitudinal axis 412/412A of the coupling lumen portion 410/410A in a distal direction. In certain embodiments the sealing surface extends at an angle θ with respect to the longitudinal axis 412/412A of the coupling lumen portion 410/410A. Such angle is between 1° and 75°, preferably between º and 20°, even more preferably between 3º and 15°. FIG. 12 is an exemplary cross-section view of a portion of applicator tip 100/100A. Angle θ is formed by longitudinal axis 412/412A and sealing surface axis 700/700A.

Applicator tips of the present disclosure are configured for attachment to a fluid dispensing device. In some embodiments the fluid dispensing device comprises a syringe. Any suitable connection may be used, for example a slip fit or friction fit connection. When used to dispense viscous fluids a secure attachment is needed to ensure that the fluid pressure does not case the applicator tip from dislodging the dispensing device. Certain embodiments include a mechanical connection, such as a threaded connection between the applicator tip and the dispensing device.

In certain embodiments, the applicator tips of the present disclosure are composed of a material(s) impenetrable to visible light. In some forms, the applicator tips of the present disclosure comprise a material which is impenetrable to ultraviolet light, or to both visible light and ultraviolet light. Such configurations are useful when dispensing a light-activated or curable fluid onto a substrate. In this way the fluid is preferentially dispersed prior to exposure to light. Accordingly, applicator tips of the present disclosure may be comprised wholly or in part of polymeric materials optionally including additives to impart light-impenetrable properties as detailed above. Such polymeric materials may be molded as one monolithic part or multiple parts for later combination. Other manufacturing operations can also be used, alone or in conjunction with molding. In additional to polymeric materials, applicator tips as described herein may comprise biocompatible metals, including but not limited to: stainless steel, titanium, titanium alloy, cobalt-chromium alloys, and magnesium alloys.

Applicator tips of the present disclosure may be configured so that upon threaded connection to a syringe the dispensing opening is positioned favorably. For example, many syringe barrels include two opposed finger flanges to allow the user to grasp the barrel while compressing the plunger. Such finger flanges extend along a flange axis on opposing sides of the barrel. Thus, in some forms, the threads of the applicator tips as described herein are calibrated so that the tip body is threadedly engageable to the syringe barrel while the flange axis extends in the same direction as the distal dispensing opening. In some forms the applicator tip is further calibrated such that the exterior surface of the cannulated seal element contacts and seals against the interior walls of the applicator tip while the flange axis extends in the same direction as the distal dispensing opening.

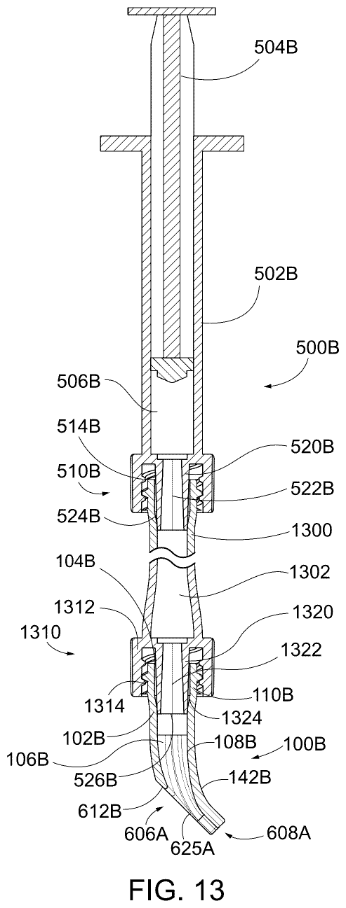

In certain embodiments the present disclosure provides a method for assembling an apparatus for dispensing a viscous material. Such method comprising threadedly connecting an applicator tip as described herein to a distal spout of a syringe barrel or to an extension tube connected to a distal spout of a syringe barrel. Where the applicator tip does not include a threaded connection, such methods comprise attaching the applicator tip to a distal spout of a syringe barrel or to an extension tube connected to a distal spout of a syringe barrel. Preferably such methods create a fluid tight seal between the syringe barrel and the applicator tip. One embodiment of such an extension tube is illustrated in FIG. 13. The illustrated embodiment includes many of the features detailed above for tip 100 and tip 100A, which are labeled with corresponding numbers followed by “B”. Briefly, applicator tip 100B defines a tip body lumen 106B extending from proximal opening 104B to anterior opening 606A and bottom opening 606A. Applicator tip 100B comprises proximal coupling portion 110B having threads 112B positioned on an external surface 114B. In the illustrated embodiment, extension tube 1300 includes tube lumen 1302 extending from syringe barrel cannula 522B to applicator tip proximal opening 104B. Extension tube 1300 comprises a distal end 1310 including connector 1312 having internal threads 1314 configured to threadedly engage with the external threads of applicator tip 100B. Connector 1312 also comprises a cannulated spout 1320 defining cannula 1322 and exterior side surface 1324. As disclosed herein, exterior side surface is configured to contact and seal against an interior surface of an applicator tip as disclosure herein. In accordance with some forms, extension tube 1300 is a rigid tube. Such extension tubes may be useful, for example, when inserting the applicator through a cannulated delivery device. In certain embodiments, extension tube 1300 comprises a material which is impenetrable to ultraviolet light, and/or visible light.

In some forms, the present disclosure provides a method for dispensing a flowable material. Such methods comprise causing a flowable material to flow from the syringe barrel and out of the dispensing opening. Any suitable force may be applied to the syringe barrel, or other dispensing device. For example, in one form pressure is applied to the flowable material be advancing a plunger within a chamber defined by the syringe barrel.

The present disclosure also provides, in certain aspects, medical kits that include an applicator tip as described herein. In certain embodiments a medical kit may comprise a flowable material for administration to a patient, or one or more precursor materials. A precursor material may comprise a dehydrated or similar material which must be hydrated to form a flowable material prior to administration. Such flowable and/or precursor materials may be packed in a sterile sealed package. Optionally, the flowable and/or precursor materials may be stored in a package which is impermeable to visible and/or ultraviolet light. In certain embodiments, the flowable material comprises a photocurable sealant or adhesive material. In accordance with some forms, the applicator tip is provided in a sealed sterile medical package. Illustratively, such packaging may include a backing layer and a front film layer that are joined by a boundary of pressure-adhesive as is conventional in medical packaging, wherein the contents of the packaging are sealed between the backing layer and front film layer. Sterilization may be achieved, for example, by irradiation, ethylene oxide gas, or any other suitable sterilization technique, and the materials and other properties of the medical packaging will be selected accordingly. The medical packaging in other aspects can include a further, outer package containing a desiccant, which can act to maintain a dry condition of the applicator tip within the inner package when that inner package is somewhat vapor permeable.

### Embodiments

The following provides an enumerated listing of some of the embodiments disclosed herein. It will be understood that this listing is non-limiting, and that individual features or combinations of features (e.g., 2, 3 or 4 features) as described in the Detailed Description above can be incorporated with the below-listed Embodiments to provide additional disclosed embodiments herein.
1. An applicator tip for connection to a syringe barrel having a distal end including a cannulated spout spaced from and surrounded by a connector having an internal connector thread on an internal wall of the connector, for dispensing a viscous material from the syringe barrel, the applicator tip comprising:
   a tip body defining a proximal opening, a dispensing opening spaced from the proximal opening, and a tip body lumen extending from the proximal opening to the dispensing opening, the tip body further including a proximal coupling portion defining an exterior thread cooperable with the internal connector thread of the syringe barrel to establish a threadedly attached condition of the applicator tip to the syringe barrel in which the tip body lumen is in fluid communication with an output opening of the cannulated spout;
   wherein the tip body lumen is defined by an interior lumen wall, and wherein the interior lumen wall includes a sealing surface portion configured to contact and seal against an exterior surface of the cannulated spout in the threadedly attached condition.
2. The applicator tip of embodiment 1, wherein the dispensing opening is a slot.
3. The applicator tip of embodiment 2, wherein the dispensing opening is a trapezoidal slot.
4. The applicator tip of any one of embodiments 1 to 3, wherein the tip body lumen includes a distal lumen portion having a longitudinal segment having a decreasing cross-sectional area as it extends distally.
5. The applicator tip of any preceding embodiment, wherein the tip body includes a distal application portion having a first exterior sidewall portion defining at least one surface that is convexly curved in a longitudinal direction along the distal application portion, optionally wherein the distal application portion further has a second exterior sidewall portion opposite the first exterior sidewall portion and defining at least one surface that is concavely curved in the longitudinal direction along the distal application portion.
6. The applicator tip of any preceding embodiment, wherein the interior lumen wall defines a decreasing lumen diameter in a distal direction that provides said sealing surface portion.
7. The applicator tip of embodiment 5, wherein the longitudinal segment having an increasing cross-sectional area extends to the dispensing opening.
8. The applicator tip of any preceding embodiment, wherein the tip body lumen includes a proximal coupling lumen portion within said proximal coupling portion that is substantially circular in cross section and has a longitudinal axis.
9. The applicator tip of embodiment 8, wherein a distal end of the tip body defines an end edge surface in a plane oblique to the longitudinal axis of the proximal coupling lumen portion.
10. The applicator tip of embodiment 8 or 9, wherein the longitudinal axis of the proximal coupling lumen portion intersects the dispensing opening.
11. The applicator tip of any preceding embodiment, wherein the tip body is composed of a material impenetrable to visible light and/or ultraviolet light.
12. The applicator tip of any preceding embodiment, wherein the tip body lumen defines a single continuous passageway through the tip body that does not intersect any other passageway in the tip body.
13. The applicator tip of any preceding embodiment, wherein a distal region of the tip body defines a fluid control member comprising a first sidewall opposite a second sidewall, and an upper wall extending between the first sidewall and the second sidewall, the upper wall opposite an open side of the fluid control member.
14. The applicator tip of embodiment 13, wherein the fluid control member is configured for positioning a longitudinally extending edge of the first sidewall and a longitudinally extending edge of the second sidewall against a target surface such that the target surface, first sidewall, second sidewall and upper wall together define a composite longitudinal lumen for receiving a bead of a fluid dispensed through the tip body.
15. A product, comprising:
   a syringe barrel having a distal end including a cannulated spout spaced from and surrounded by a connector having internal connector threads on an internal wall of the connector; and
   an applicator tip of any one of embodiments 1 to 14 threadedly connected to or threadedly connectable to the syringe barrel to establish the threadedly connected condition.
16. A product, comprising:
   a syringe barrel having a distal end including a cannulated spout;
   an applicator tip comprising a tip body defining a proximal opening, a dispensing opening spaced from the proximal opening, and a tip body lumen extending from the proximal opening to the dispensing opening, the tip body having an interior lumen wall defining the tip body lumen; and
   the applicator tip being threadedly connected to or threadedly connectable to the syringe body to establish a connected condition in which the tip body lumen is in fluid communication with a distal end opening of the cannulated spout and in which a sealing surface portion of the interior lumen wall is in contact with and sealed against an exterior side surface of the cannulated spout.
17. The product of embodiment 16, wherein the cannulated spout includes a distally-tapering cone.
18. The product of embodiment 16 or 17, wherein the distal end of the syringe barrel includes a connector having connector threads on an internal wall thereof, with the cannulated spout being spaced from and surrounded by the connector; and
   the tip body includes a proximal coupling portion defining an exterior thread that is threadably cooperable with the connector threads.
19. The product of embodiment 18, wherein the tip body lumen includes a distal lumen portion having a decreasing cross-sectional area as it extends distally.
20. The product of embodiment 18 or 19, wherein the sealing surface occurs on a portion of the interior lumen walls that define a decreasing lumen diameter in a distal direction.
21. The product of embodiment 20, wherein the sealing surface occurs distal to the proximal coupling portion of the tip body.
22. The product of any one of embodiments 16 to 21, wherein the tip body includes a distal application portion having an exterior sidewall portion defining at least one surface that is convexly curved in a longitudinal direction along the distal application portion.
23. The product of any one of embodiments 16 to 22, wherein the dispensing opening is a slot.
24. The product of embodiment 23, wherein the dispensing opening is a trapezoidal slot.
25. The product of any one of embodiments 16 to 24, wherein a distal end of the tip body defines an end edge surface in a plane oblique to the longitudinal axis of the proximal coupling portion.
26. The product of any one of embodiments 16 to 25, wherein the longitudinal axis of the proximal coupling portion intersects the dispensing opening.
27. The product of any one of embodiments 16 to 26, wherein the tip body is composed of a material impenetrable to visible light and/or ultraviolet light.
28 The product of any one of embodiments 16 to 27, wherein said tip body lumen is the only lumen through the tip body.
29. The product of any one of embodiments 16 to 28, wherein the tip body defines a fluid control member comprising a first sidewall opposite a second sidewall, and an upper wall extending between the first sidewall and the second sidewall, the upper wall opposite an open side of the fluid control member.
30. The product of any one of embodiments 16 to 29, wherein the distal dispensing opening extends in a first direction, wherein the syringe barrel comprises proximal first and second opposed finger flanges extending along a flange axis, and wherein the flange axis extends in the first direction.
31. The product of any one of embodiments 16 to 30, which is an assembled apparatus in which the applicator tip is in the threadedly connected condition.
32. The product of any one of embodiments 16 to 30, which is a kit including the syringe barrel and the applicator tip not in the threadedly connected condition.
33. An applicator tip for dispensing a viscous material, the applicator tip comprising:
   a tip body defining a proximal opening, a dispensing opening spaced from the proximal opening, and a tip body lumen extending from the proximal opening to the dispensing opening;
   the tip body having a distal region that defines a fluid control member comprising a first sidewall opposite a second sidewall, and an upper wall extending between the first sidewall and the second sidewall, the upper wall opposite an open side of the fluid control member.
34. The applicator tip of embodiment 33, wherein the fluid control member is configured for positioning a longitudinally extending edge of the first sidewall and a longitudinally extending edge of the second sidewall against a target surface such that the target surface, first sidewall, second sidewall and upper wall together define a composite longitudinal lumen for receiving a bead of a fluid dispensed through the tip body.
35. The applicator tip of any one of embodiments 33 or 34, wherein the tip body lumen is defined by a tip body lumen wall, and wherein the upper wall has an inner surface contiguous with the tip body lumen wall and extending to the dispensing opening, and wherein the inner surface is a smooth surface.
36. The applicator tip of any one of embodiments 33 to 35, wherein the tip body lumen comprises a coupling lumen portion having a substantially circular cross-sectional area.
37. The applicator tip of embodiment 36, wherein the interior lumen wall defines a decreasing lumen diameter in a distal direction that provides a sealing surface portion.
38. The applicator tip of any one of embodiments 33 to 37, wherein the tip body is composed of a material impenetrable to visible light and/or ultraviolet light.
39. The applicator tip of any one of embodiments 33 to 38, wherein the tip body includes a distal application portion having a first exterior sidewall portion defining at least one surface that is convexly curved in a longitudinal direction along the distal application portion.
40. A product, comprising:
   a syringe barrel having a distal end with a cannulated spout; and
   an applicator tip according to any one of embodiments 33 to 39 connected to or connectable to the syringe barrel to establish a connected condition in which the tip body lumen is in fluid communication with an opening of the cannulated spout.
41. The product of embodiment 40, which is an assembled apparatus in which the applicator tip is in the connected condition.
42. The product of embodiment 40, which is a kit including the syringe barrel and the applicator tip not in the connected condition.
43. The product of embodiment 41, wherein the product is contained within a sterile sealed package.
44. A method for assembling an apparatus for dispensing a flowable material, comprising:
   connecting an applicator tip according to any one of embodiments 1 to 14 or any one of embodiments 33 to 39 to a syringe barrel or to an extension tube connected to a syringe barrel.
45. A method for dispensing a flowable material, comprising:
   providing a product of any one of embodiments 15 to 32, a product of any one of embodiments 40 to 43, or an apparatus assembled according to embodiment 44; and
   causing the flowable material to flow from the syringe barrel and out of the dispensing opening.
46. The method of embodiment 45, wherein said causing includes advancing a plunger within a chamber defined by the syringe barrel.
47. A medical kit for administration of a flowable material to a patient, comprising:
   a flowable material for administration to a patient, or one or more precursor materials for preparing a flowable material for administration to a patient; and
   an applicator tip according to any one of embodiments 1 to 14, a product according to any one of embodiments 15 to 32, an applicator tip according to any one of embodiments 33 to 39, or a product according to any one of embodiments 40 to 43.
48. The medical kit of embodiment 47, wherein the flowable material is a photocurable sealant or photocurable adhesive material.

The uses of the terms “a” and “an” and “the” and similar references herein (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the products or methods defined by the claims.

While embodiments of the disclosure have been illustrated and described in detail in the drawings and foregoing description, the same is to be considered as illustrative and not restrictive in character, it being understood that only some embodiments have been shown and described and that all changes and modifications that come within the spirit of the disclosures herein are desired to be protected.

1. An applicator tip for connection to a syringe barrel having a distal end including a cannulated spout spaced from and surrounded by a connector having an internal connector thread on an internal wall of the connector, for dispensing a viscous material from the syringe barrel, the applicator tip comprising:
a tip body defining a proximal opening, a dispensing opening spaced from the proximal opening, and a tip body lumen extending from the proximal opening to the dispensing opening, the tip body further including a proximal coupling portion defining an exterior thread cooperable with the internal connector thread of the syringe barrel to establish a threadedly attached condition of the applicator tip to the syringe barrel in which the tip body lumen is in fluid communication with an output opening of the cannulated spout;
wherein the tip body lumen is defined by an interior lumen wall, and wherein the interior lumen wall includes a sealing surface portion configured to contact and seal against an exterior surface of the cannulated spout in the threadedly attached condition.

2. The applicator tip of claim 1, wherein the dispensing opening is a slot.

3. The applicator tip of claim 2, wherein the dispensing opening is a trapezoidal slot.

4. The applicator tip of claim 1, wherein the tip body lumen includes a distal lumen portion having a longitudinal segment having a decreasing cross-sectional area as it extends distally.

5. The applicator tip of claim 1, wherein the tip body includes a distal application portion having a first exterior sidewall portion defining at least one surface that is convexly curved in a longitudinal direction along the distal application portion, optionally wherein the distal application portion further has a second exterior sidewall portion opposite the first exterior sidewall portion and defining at least one surface that is concavely curved in the longitudinal direction along the distal application portion.

6. The applicator tip of claim 1, wherein the interior lumen wall defines a decreasing lumen diameter in a distal direction that provides said sealing surface portion.

7. The applicator tip of claim 4, wherein the longitudinal segment having a decreasing cross-sectional area extends to the dispensing opening.

8. The applicator tip of claim 1, wherein the tip body lumen includes a proximal coupling lumen portion within said proximal coupling portion that is substantially circular in cross section and has a longitudinal axis.

9. The applicator tip of claim 8, wherein a distal end of the tip body defines an end edge surface in a plane oblique to the longitudinal axis of the proximal coupling lumen portion.

10. The applicator tip of claim 8, wherein the longitudinal axis of the proximal coupling lumen portion intersects the dispensing opening.

11. The applicator tip of claim 1, wherein the tip body is composed of a material impenetrable to visible light and/or ultraviolet light.

12. The applicator tip of claim 1, wherein the tip body lumen defines a single continuous passageway through the tip body that does not intersect any other passageway in the tip body.

13. The applicator tip of claim 1, wherein a distal region of the tip body defines a fluid control member comprising a first sidewall opposite a second sidewall, and an upper wall extending between the first sidewall and the second sidewall, the upper wall opposite an open side of the fluid control member.

14. The applicator tip of claim 13, wherein the fluid control member is configured for positioning a longitudinally extending edge of the first sidewall and a longitudinally extending edge of the second sidewall against a target surface such that the target surface, first sidewall, second sidewall and upper wall together define a composite longitudinal lumen for receiving a bead of a fluid dispensed through the tip body.

15. A product, comprising:
a syringe barrel having a distal end including a cannulated spout spaced from and surrounded by a connector having internal connector threads on an internal wall of the connector; and
an applicator tip of claim 1 threadedly connected to or threadedly connectable to the syringe barrel to establish the threadedly connected condition.

16. (canceled)

17. The product of claim 15, wherein the cannulated spout includes a distally-tapering cone.

18. The product of claim 15, wherein the distal end of the syringe barrel includes a connector having connector threads on an internal wall thereof, with the cannulated spout being spaced from and surrounded by the connector; and
the tip body includes a proximal coupling portion defining an exterior thread that is threadably cooperable with the connector threads.

19-30. (canceled)

31. The product of claim 15, which is an assembled apparatus in which the applicator tip is in the threadedly connected condition.

32. The product of claim 15, which is a kit including the syringe barrel and the applicator tip not in the threadedly connected condition.

33. An applicator tip for dispensing a viscous material, the applicator tip comprising:
a tip body defining a proximal opening, a dispensing opening spaced from the proximal opening, and a tip body lumen extending from the proximal opening to the dispensing opening;
the tip body having a distal region that defines a fluid control member comprising a first sidewall opposite a second sidewall, and an upper wall extending between the first sidewall and the second sidewall, the upper wall opposite an open side of the fluid control member.

34. The applicator tip of claim 33, wherein the fluid control member is configured for positioning a longitudinally extending edge of the first sidewall and a longitudinally extending edge of the second sidewall against a target surface such that the target surface, first sidewall, second sidewall and upper wall together define a composite longitudinal lumen for receiving a bead of a fluid dispensed through the tip body.

35. The applicator tip of claim 33, wherein the tip body lumen is defined by a tip body lumen wall, and wherein the upper wall has an inner surface contiguous with the tip body lumen wall and extending to the dispensing opening, and wherein the inner surface is a smooth surface.

36. The applicator tip of claim 33, wherein the tip body lumen comprises a coupling lumen portion having a substantially circular cross-sectional area.

37. The applicator tip of claim 36, wherein the interior lumen wall defines a decreasing lumen diameter in a distal direction that provides a sealing surface portion.

38. The applicator tip of claim 33, wherein the tip body is composed of a material impenetrable to visible light and/or ultraviolet light.

39. The applicator tip of claim 33, wherein the tip body includes a distal application portion having a first exterior sidewall portion defining at least one surface that is convexly curved in a longitudinal direction along the distal application portion.

40. A product, comprising:
a syringe barrel having a distal end with a cannulated spout; and
an applicator tip according to claim 33 connected to or connectable to the syringe barrel to establish a connected condition in which the tip body lumen is in fluid communication with an opening of the cannulated spout.

41. The product of claim 40, which is an assembled apparatus in which the applicator tip is in the connected condition.

42. The product of claim 40, which is a kit including the syringe barrel and the applicator tip not in the connected condition.

43. The product of claim 41, wherein the product is contained within a sterile sealed package.

44. A method for assembling an apparatus for dispensing a flowable material, comprising:
connecting an applicator tip according to claim 1 to a syringe barrel or to an extension tube connected to a syringe barrel.

45. A method for dispensing a flowable material, comprising:
providing a product of claim 15; and
causing the flowable material to flow from the syringe barrel and out of the dispensing opening.

46. The method of claim 45, wherein said causing includes advancing a plunger within a chamber defined by the syringe barrel.

47. A medical kit for administration of a flowable material to a patient, comprising:
a flowable material for administration to a patient, or one or more precursor materials for preparing a flowable material for administration to a patient; and
an applicator tip according to claim 1.

48. The medical kit of claim 47, wherein the flowable material is a photocurable sealant or photocurable adhesive material.
